(19) United States
(12) Patent Application Publication
(10) Pub. No.: US 2005/0021032 A1
(43) Pub. Date: Jan. 27, 2005

(54) Cervical spine fixator and screwdriver used therefor

(76) Inventor: Ja-Kyo Koo, Seoul (KR)

Correspondence Address: DICKSTEIN SHAPIRO MORIN & OSHINSKY LLP, 2101 L Street, NW, Washington, DC, 20037, US

(21) Appl. No.: 10/890,183

(22) Filed: Jul. 14, 2004

(30) Foreign Application Priority Data
Jul. 22, 2003 (KR) 10-2003-00050152

(51) Int. Cl. A61B 17/56

(52) U.S. Cl. 606/69

(57) ABSTRACT

A cervical spine fixator and a screwdriver used therefore is disclosed wherein the fixator comprises plates (12, 212) for being adjoined to a cervical spine fixation region and a plurality of screws (14, 214). The plurality of screws are fixedly inserted into a patient's cervical spine and simultaneously coupled to the plates, wherein an adjacent region coupled with the screws in the plates is fixed with screw head fixture members (16, 216) having a protruder, which is deformed under the effect of pressure by a screw head when the screws are inserted and returns to their position of origin when the screw head is completely accommodated into the plates to be hitched by the screw head. The plates are formed thereon with receiving grooves (22, 222) for accommodating the screw head and the screw head fixture member. The receiving grooves are formed with openings into which the screws are inserted and a recess into which the screw head is received. The screw (214) formed with a groove of concaved curvature is installed or removed by a screwdriver (230) formed with a protruder of convex curvature, such that it is easy to manufacture, and the thickness of the plates is reduced, thus eliminating the feeling of a foreign substance after a surgical operation.

### FIELD OF THE INVENTION

The present invention relates generally to a cervical spine fixator as an anterior cervical implant used for treating and correcting cervical disc diseases, cervical hernial disc diseases, cervical spondylosis myelopathy, cervical fractures, dislocations, tumors and cervical kyphotic deformity in orthopedics and neurosurgical fields.

### BACKGROUND OF THE INVENTION

In general, cervical spine disorders are treated by holding respective cervical vertebrae in desired spatial relationships and orientations relative to each other. One common device for spinal fixation includes a plate and a fixation device comprising a plurality of screws for being fixedly inserted to the plate.

The fixation device includes fixation screws for being driven or fastened into the upper and lower cervical vertebrae of the cervical spine, whereby respective vertebrae are fixed and treated by the locking force of the plates and the fixed screws.

A surgical operation by the fixation device is conducted as follows. A surgeon makes an incision into a patient's affected part of the cervical vertebrae. Next, while the plate of the fixation device is adjoined to the affected part, the screw is inserted into the front surface of the plate to allow the fixation device to be fixed to the cervical vertebrae positioned at the upper and lower ends of the affected part.

However, there is a drawback associated with this technique in that screws or other fasteners tend to gradually become loose after fixation. Receiving a slight shock or vibration due to walking, climbing stairs or engaging in a vigorous activity following treatment increases this tendency, jeopardizing the integrity of the fixation. Moreover, as the fasteners are loosened, the outward protrusion of the heads over other components of the fasteners can be a source of discomfort and potentially cause trauma to adjacent and surrounding soft tissue, resulting in a deformity of the affected part.

There have been attempts to solve the above drawback. For example, an auxiliary plate hitching the head of the screw is locked by a fastening member after the screw is fixed, or devices such as a cam locking system and the like for preventing the screws from backing out have been developed.

However, there is a drawback in the structure of fixing the auxiliary plate by the fastening member to thereby prevent the screws from backing out according to the prior art thus described in that the auxiliary plate can become loose due to the unscrewing of the fastening member. There is still another drawback in that it is inconvenient to conduct an operation due to the re-fixation of a separate fastening member after the fixation of the screws. There is still a further drawback in that the cam locking system is too complicated in structure thereof.

Meanwhile, other attempts have been disclosed in WO 00/78238A1 and EP 1169971A2, where a member elastically deformed by the effect of pressure is used to fix a head of a screw.

According to WO 00/78238A1, at least one retaining means is provided which can be elastically deformed under the effect of pressure enabling a screw to pass into and be installed in a bore hole, whereby the retaining means returns to its position of origin and is not deformed when the effect of the pressure is withdrawn, being positioned above a head of the screw. The retaining means is received into a separate bore hole adjacent to the bore hole accommodating the screw.

EP 1169971A2 discloses an implant, particularly for the spinal column, comprising a joining member such as a plate exhibiting openings or orifices, bone-anchoring members such as bone screws capable of being accommodated in the orifices and at least one split ring capable of holding the members in the orifices. The split ring can come into direct contact with the anchoring member or members to hold the member or members in the orifices.

However, there are drawbacks in WO 00/78238A1 and EP 1169971A2 in that the retaining means or split ring is accommodated in the orifices of the plate by a member elastically deformed by the effect of pressure for fixing of a screw head, making the fixation process more difficult, creating more difficulty in manufacturing, and resulting in a thickened plate. When the plate is thickened, a patient may feel a foreign substance after a surgical operation. Thus, it has been suggested by patients and surgeons that the plate be thinned to remove the discomfort and the cervical spine fixator be simple in operation to reduce the operating time.

### SUMMARY OF THE INVENTION

The present invention is disclosed to meet the above-mentioned requirements and it is an object of the present invention to provide a cervical spine fixator that is easier to manufacture and a plate that is reduced in thickness, thus decreasing the feeling of a foreign substance after a surgical operation.

It is another object of the present invention to provide a screwdriver for a cervical spine fixator adapted to be convenient for surgery and to shorten the operating time.

The cervical spine fixator according to one embodiment of the present invention comprises: a plate for being adjoined to a cervical spine fixation region; and a plurality of screws for insertion and fixation in the cervical spine and simultaneously coupled to the plate. An adjacent region coupled with the screws in the plate is fixed with a screw head fixture member having a protruder, which is deformed under the effect of pressure by a screw head when the screw is inserted and returns to its position of origin when the screw head is completely accommodated into the plate. Said plate is formed thereon with a receiving groove for accommodating the screw head and the screw head fixture member. The receiving groove is formed with openings into which the screws are inserted and a recess into which the screw head is received.

The screw head fixture member is an elastic plate protrusively formed with the protruder.

Preferably, the screw head fixture member is a U-shaped elastic ring functioning as the protruder. Preferably, the distal tip ends of the protruder or the elastic ring bend towards mutually opposite directions. Preferably, the protruder or the U-shaped elastic ring is tapered-off in the cross-section thereof towards the opposite side of the distal tip end so as to be easily deformed.

Preferaby, the distal tip ends of the protruder or the U-shaped elastic ring is formed with a hitching protruder, and the recess of the plate is formed with a hitching jaw where the hitching protruder is hitched, and the hitching jaw is a hitching hole through which the plate passes.

Furthermore, the mid-section of the plate is formed with a window-shaped hole to observe a cage between the vertebrae during surgery, and the window-shaped hole is formed with threads into which a screw is threaded for fixing the cage.

The screw head has a cornical-shaped incline plane configuration so that the protruder or the U-shaped elastic ring can smoothly slide to be elastically deformed under the effect of pressure when the screws are inserted into the plate.

The protruder of the screw head fixture member or an opposite end of the distal end at the U-shaped elastic ring is fixed to the plate by a rivet.

The upper surface of the screw head is grooved in the shape of a straight line or a cross, and the grooved surface is deepened towards the center thereof to form a concaved curvature so that the protruder or the U-shaped elastic ring can be deformed under the effect of pressure by the tip end of a screwdriver when the screws are installed or removed.

The distal tip end of the screwdriver for rotating the screw is protrusively formed with a protruder in the shape of a straight line or a cross so as to be inserted into the groove, whereas the protruder is formed in a convex shape for being adjoined onto the concave curvature of the screw head.

### BRIEF DESCRIPTION OF THE DRAWINGS

For a better understanding of the nature and objects of the present invention, reference should be made to the following detailed description with the accompanying drawings, in which:

FIG. 1 is a perspective view for illustrating a cervical spine fixator according to a first embodiment of the present invention;

FIG. 2 is an exploded perspective view of FIG. 1;

FIG. 3 is a cross-sectional view taken along line A-A of FIG. 1;

FIG. 4 is a plan for illustrating a deformed state of a screw head fixture member when a screw is inserted;

FIG. 5 is a cross-sectional view for illustrating a screw head fixture member hitching a screw head when a screw is completely inserted according to the present invention;

FIG. 6 is a perspective view for illustrating a cervical spine fixator according to a second embodiment of the present invention;

FIG. 7 is a cross-sectional view taken along the arrow B-B of FIG. 6;

FIG. 8 is a perspective view of a cervical spine fixator according to a third embodiment of the present invention;

FIG. 9 is an exploded perspective view of FIG. 8;

FIG. 10 is a bottom view taken from a lower side of the cervical spine fixator of FIG. 8; and

FIG. 11 is a perspective view for illustrating a screwdriver used for a cervical spine fixator during surgery according to the present invention.

### DETAILED DESCRIPTION OF THE PREFERRED EMBODIMENT

The preferred embodiments of the present invention will now be described in detail with reference to the annexed drawings, where the present embodiments are not limiting the scope of the present invention but are given only as an illustrative purpose.

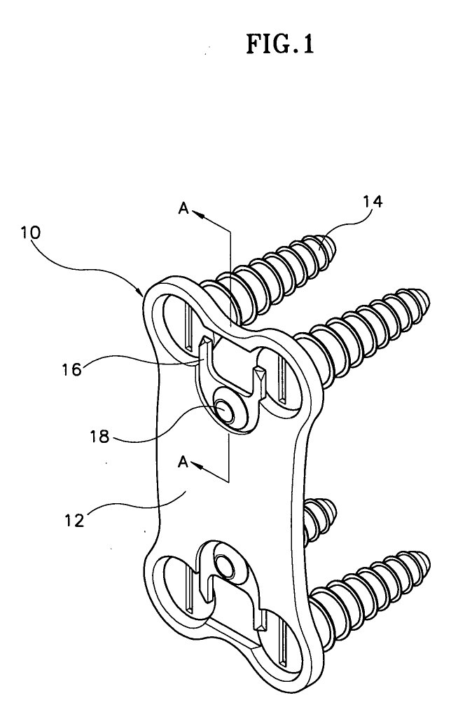

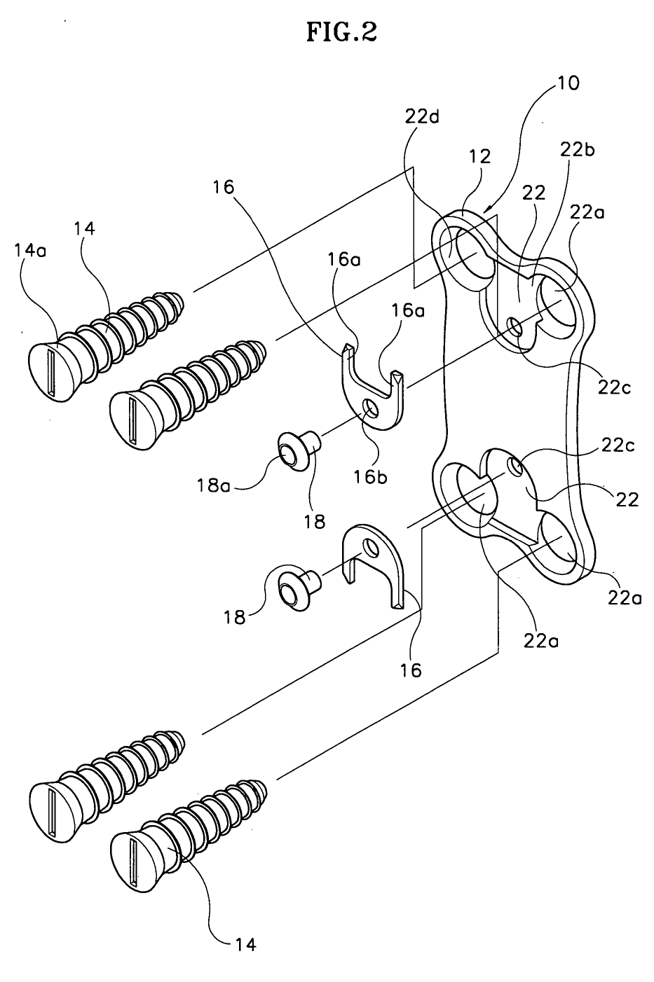

As shown in FIGS. 1 and 2, a cervical spine fixator (10) according to a first embodiment of the present invention includes a plate (12) for being adjoined to a cervical spine fixture region, a plurality of screws coupled to the plate (12) and simultaneously fastened to the cervical spine, and a screw head fixture member (16) fixed to the plate (12) for preventing the screws from backing out.

The screw head fixture member (16) is fixed to the plate (12) by way of a well-known attachment method such as riveting (18) or welding. Generally, the plate (12) has a rectangular shape and is bent to conform to the curvature of the cervical spine. Receiving grooves are formed on the surface of the plate (12) opposite to the surface that is affixed to the cervical spine, for accommodating the head of the screw (14) and the screw head fixture member (16).

The receiving grooves (22) are formed at both ends of the plate (12), and each receiving groove (12) has an approximate T-shape when viewed from the top of the plate (12). Both lateral ends of the groove (22) are formed with openings (22a, 22a) for insertion of the screws. The vertical portion of the T-shaped groove (22) is formed with a recess (22b) for accommodating the screw head fixture member (16), and the recess (22b) has a rivet hole (22c) at one end thereof for insertion of a rivet (18) for fixing the screw head fixture member (16).

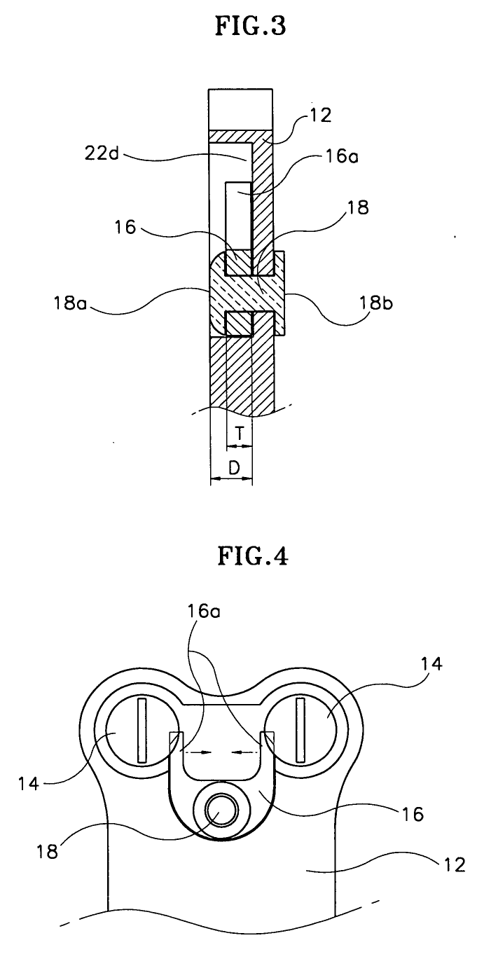

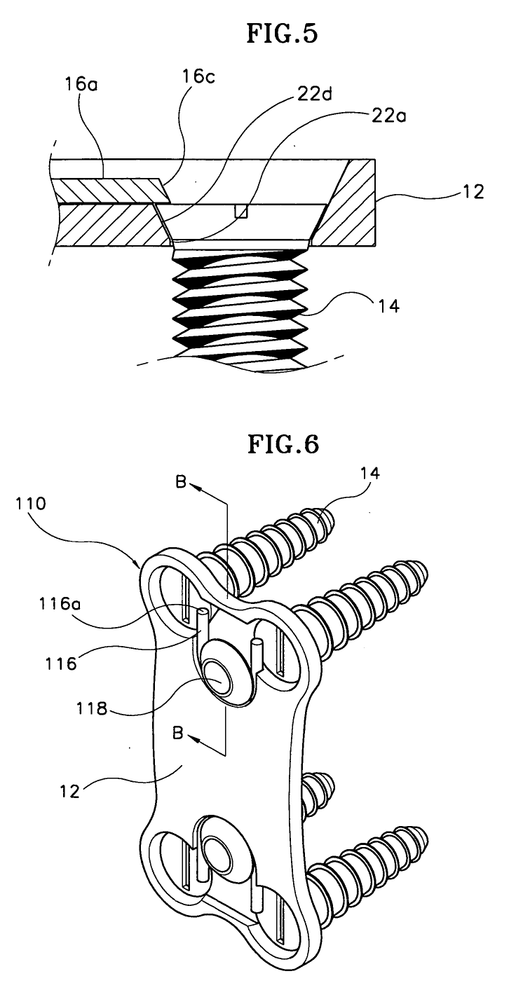

Furthermore, as illustrated in FIG. 3, the depth (D) of the recess (22b) is equal to or deeper than the thickness (T) of the screw head fixture member (16) such that a head (18a) of the rivet (18) does not protrude out of the plane surface of the plate (12). As shown in FIG. 5, the opening (22a) of the receiving groove (22) is formed with an incline (22d) to allow a head of the screw (14) to be hitched thereat.

The four screws (14) are members inserted into four holes formed at the plate (12), and the head of each screw (14) is formed with an incline (14a) for easily deforming protruders (described later) of the screw head fixture member (16) when the screw (14) is inserted.

The screw head fixture member (16) is an elastic horse shoe-shaped plate having protruders (16a) each hitching the heads of the screws (14). The screw head fixture member (16) is deformed under the effect of pressure by the incline (14a) of the heads of the screws when the screws are inserted and returns to its position of origin when the heads of the screws (14) are completely accommodated into the incline (22d) formed at the hole of the plate (12). The protruder (16a) of the screw head fixture member (16) is formed at the other side thereof with a rivet hole (16b) so that the rivet (18) is fixedly inserted into the plate (12). Each protruder (16a) is like a prong of a horse shoe magnet.

As seen in FIG. 5, the distal end of each protruder (16a) is formed with an incline (16c) for allowing the incline (14a) of the screw to smoothly slide when the head of the screw (14) is inserted and for prompting the protruder (16a) to be easily deformed. The protruder (16a) tapers off towards the opposite side of the distal end so as to be easily deformed during surgery.

The rivet (18) is inserted into the rivet hole (16b) of the screw head fixture member and the rivet hole (22c) of the plate, and as seen in FIG. 3, the rivet (18) forms a heading (18b) at the surface of the plate (12) that is affixed to the cervical spine to thereby fix the screw head fixture member (16).

In the cervical spine fixator according to the first embodiment of the present invention thus constructed, the screw (14) is inserted into the opening (22a) of the plate (12), where the head of the screw (14) is accommodated into the incline (22d) of the hole (22a) to slide along the incline (16c) formed at the protruder (16a) of the screw head fixture member (16), and as shown in FIG. 4, the head of the screw (14) pushes the protruder (16a) in the arrow direction.

Furthermore, when the head of the screw (14) is completely fitted at the incline (22d), the protruder (16a) returns to its original position to be hitched by the upper surface of the head of the screw (14) as shown in FIG. 5, thus preventing the screw from being released or loosened.

FIG. 6 is a perspective view for illustrating a cervical spine fixator according to a second embodiment of the present invention.

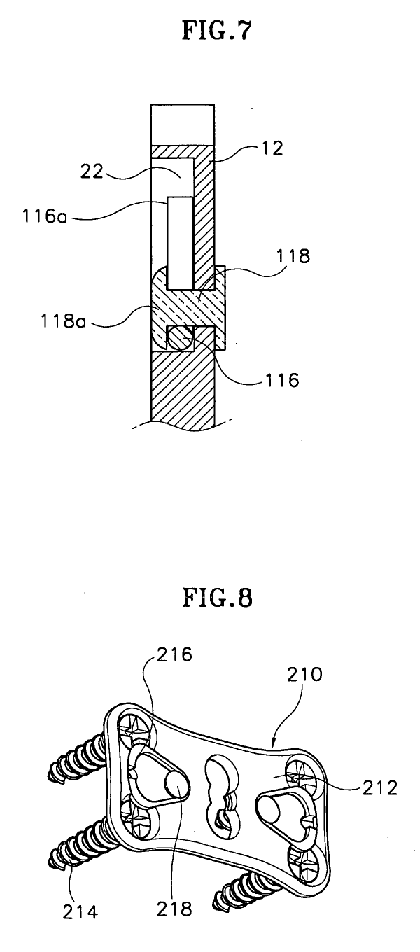

The cervical spine fixator (110) according to the second embodiment of the present invention includes a screw head fixture member (116) of a U-shaped ring, and both ends (116a) of the U-shaped ring function as the protruders of the first embodiment. The rivet (118) of the present invention has a head large enough to press the curvature of the U-shaped ring. FIG. 7 is a cross-sectional view taken along an arrow of FIG. 6. Other constructions of the second embodiment are the same as those of the first embodiment such that further explanation will not be necessary.

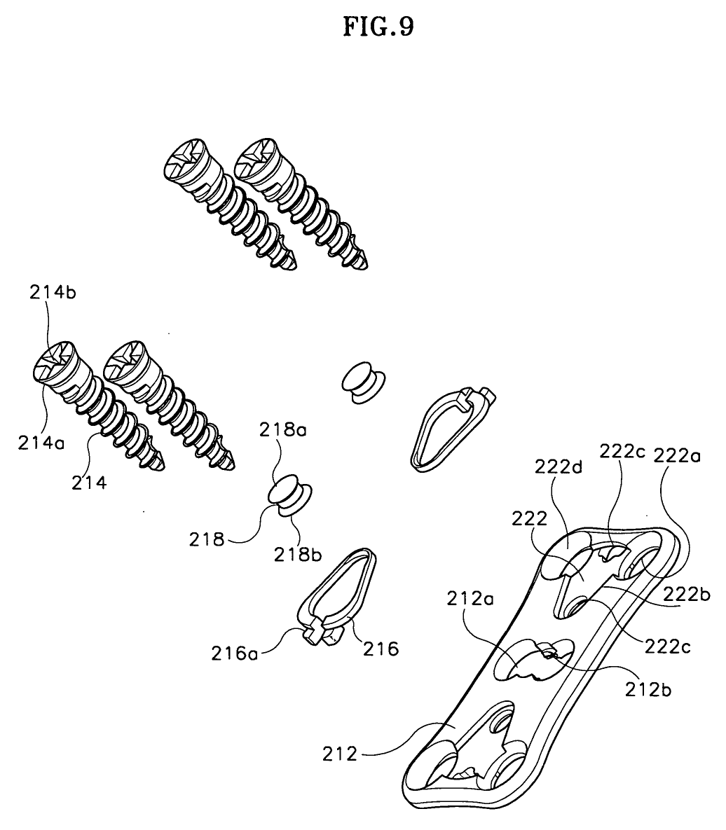

FIGS. 8 and 9 are respectively a perspective view and an exploded perspective view of a cervical spine fixator according to a third embodiment of the present invention, wherein the cervical spine fixator (210) includes a plate (212) for being attached to a cervical spine fixture region, a plurality of screws (214) coupled to the plate (212) and simultaneously fixedly inserted into the cervical spine, and a screw head fixture member (216) fixed to the plate (212) for preventing the screws (214) from being loosened. The screw head fixture member (216) is fixed to the plate (212) by way of a well-known coupling method such as riveting (218), welding or the like.

The plate (212) has a near rectangular shape and is bent to conform to the curvature of the cervical spine. The surface of the plate (212) opposite to the surface that is affixed to the cervical spine is formed with receiving grooves (222) for accommodating the screw head fixture member (216) and heads of the screws (214).

The receiving grooves (222) are formed at both ends of the plate (212), and each receiving groove (222) has an approximate T-shape when viewed from the top of the plate (212). Both lateral ends of each T-shaped groove (222) are formed with openings (222a, 222a) for insertion of the screws. The vertical portion of each T-shaped groove (222) is formed with a recess (222b) for accommodating the screw head fixture member (216), and the recess (222b) is defined at one side thereof with a rivet hole (222c) for insertion of a rivet (218) for fixing the screw head fixture member (16). The structure of accommodating the screw head fixture member (216) into the recess (222b) can markedly reduce the thickness of the plate (212), actually up to a 1.6 mm.

The depth (D) of the recess (222b) is equal to or deeper than the thickness (T) of the screw head fixture member (216) such that the head (218a) of the rivet (218) does not protrude out of the plane surface of the plate (212) as shown in FIG. 3. Furthermore, the opening (222a) of the receiving groove (222) is formed with an incline (222d) to allow the head of each screw (214) to be hitched thereat as shown in FIG. 5.

A hitching jaw is formed between the two openings (222a, 222a) in the recess (222b) where a hitching protruder of the screw head fixture member can be hitched thereat. The hitching jaw is a hitching hole (222e) that passes through the thickness of the plate (212). The width of the recess (222b) becomes narrowed from the hitching hole (222e) towards the rivet hole (222c).

Furthermore, the mid-section of the plate (212) is formed with a window-shaped hole (212a) to observe a cage between the vertebrae during surgery, and the window-shaped hole (212a) is a long hole in the width direction of the plate. The mid-section of the window hole (212a) is wasp-waisted, where a screw thread (212b) is formed with threads into which a screw (not shown) is threaded for fixing the cage.

The screws (214) are members respectively driven into the cervical spine by being inserted into four holes each formed at the plate (212).

The screw head is laterally formed with a conical incline (214a) into which the screws (214) are inserted, and the distal end of the U-shaped elastic ring (described later) of the screw head fixture member (216) can be easily deformed.

The upper surface of the screw head is formed with a crossed groove (214b), and the crossed groove (214b) becomes deepened towards the center thereof to form a concaved curvature so that the distal end of the U-shaped elastic ring (described later) of the screw head fixture member (216) can be deformed under the effect of pressure by the tip end of a screwdriver when the screws (214) are installed or removed. The upper surface of the screw head may be formed with a straight groove, which becomes deepened towards the center thereof.

The screw head fixture member (216) is a U-shaped elastic ring which functions as the protruders of the first embodiment at both ends thereof, and a gap between both ends is narrowed towards the opposite side of the distal end to fit into the width of the recess (222b) of the receiving groove. The distal ends of the screw head fixture member (216) of the U-shaped elastic ring are bent towards each other, and each bent end is formed with a hitching protruder (216a), which hitchingly fits into the hitching hole (222e).

The screw head fixture member (216) is deformed under the effect of pressure by the incline (214a) of the screw head in relation to the insertion of the screw (214) and returns to its position of origin to get hitched at the head of the screw (214) when the head of the screw (214) is completely accommodated into the incline (222d) formed at the hole of the plate (212), where the thickness or cross-section becomes smaller from one distal end of the screw head fixture member (216) to the opposite end to expedite the deformation.

The rivet (218) is hitched at an inner central portion of the screw head fixture member (216) and is simultaneously inserted into the rivet hole (222c) of the plate, and the rivet (218) is formed with a heading (218b) at a surface of the plate (212) for being attached to the cervical spine to thereby fix the screw head fixture member (216).

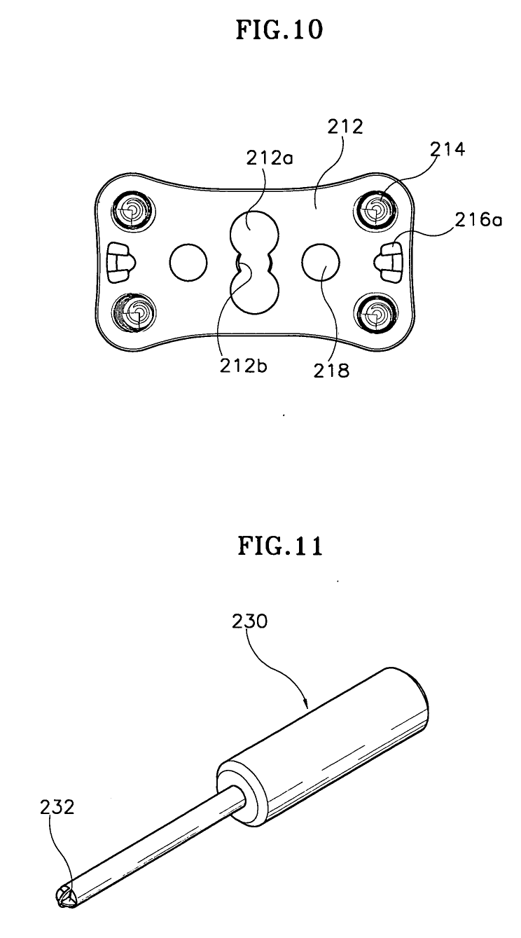

The screw head fixture member (216) is fixed by the rivet (218) after the hitching protruder (216a) is inserted into the hitching hole (222e) of the plate (212). The hitching protruder (216a) of the screw head fixture member (216) hitches and protrudes toward the surface adjoining the cervical spine at the plate (212) as shown in FIG. 10.

As a result, even if the screw becomes loose by the activity of a patient, the hitching protruder (216a) is hitched by the hitching hole (222e) of the plate (212) to prevent the screw head fixture member (216) from being protrusively deformed, thereby preventing irritation in the affected area.

In the cervical spine fixator thus described according to the third embodiment of the present invention, the screw (214) is inserted into the opening (222a) of the plate (212) and thereafter inserted into the cervical spine. The head of the screw (214) is correspondingly fitted into the incline (222d) of the hole (222a) while pushing the distal end of the screw head fixture member inward in the process.

Furthermore, when the head of the screw (214) is fully accommodated into the incline (222d), the distal end of the screw head fixture member (216) returns to its position of origin to be hitched at the upper surface of the head of the screw (214) as illustrated in FIG. 8.

FIG. 11 is a perspective view for illustrating a screwdriver for use in surgery in connection with the cervical spine fixator according to the third embodiment of the present invention. The distal end of the screwdriver (230) is formed with a crossed protruder (232) for being fitted into the crossed groove (214b) of the screw (214). The crossed protruder (232) is made of a convex curvature to be fitted into the concave curvature of the crossed groove (214b). The distal end of the screwdriver is formed with a straight protruder, which protrudes from the cross-section thereof. The straight protruder may be of a convex curvature for being fitted into the concave curvature of the straight groove of the screw.

The screwdriver thus described slowly pushes the distal end of the screw head fixture member (216) and bends the distal end of the screw head fixture member (216) when the crossed protruder (232) of the screwdriver is driven into the crossed groove (214b) of the screw (214) while the screw (214) is fixed by the screw head fixture member (216). As a result, a separate tool is not necessary for deforming the screw head fixture member (216) when the screw (214) is removed, thus shortening the operating time and adding convenience to the surgical procedure.

As apparent from the foregoing, there is an advantage in the cervical spine fixator and a screwdriver used therefor thus described according to the embodiments of the present invention in that it is easy to manufacture, and the plate is reduced in thickness, thus eliminate the feeling of a foreign substance after a surgical operation.

There is another advantage in that it is easy to perform surgery with the fixator and it is possible to shorten the operating time.

The foregoing description of the preferred embodiments of the present invention has been presented for the purpose of illustration and description. It is not intended to be exhaustive or to limit the invention to the precise form disclosed, and modifications and variations are possible in light of the above teachings or may be acquired from practice of the invention. It is intended that the scope of the invention be defined by the claims appended hereto, and their equivalents.

1. A cervical spine fixator, comprising:
a plate for being adjoined to a cervical spine fixation region; and
a plurality of screws for insertion and fixation into a cervical spine and for being simultaneously coupled to said plate, wherein an adjacent region coupled with said screws in the plate is fixed with a screw head fixture member having a protruder, which is bent under the effect of pressure by a screw head when said screw is inserted and the protruder returns to its original position when said screw head is completely accommodated into the plate to be hitched by said screw head, and said plate is formed thereon with a receiving groove for accommodating said screw head and said screw head fixture member, and said receiving groove is formed with openings for insertion of said screws and a recess for receiving said screw head.

2. The fixator as defined in claim 1, wherein said screw head fixture member is an elastic plate protrusively formed with said protruder.

3. The fixator as defined in claim 1, wherein said screw head fixture member is a U-shaped elastic ring whose both ends functioning as protruders.

4. The fixator as defined in claim 2 or 3, wherein the distal tip ends of said protruder or said elastic ring are bent towards each other.

5. The fixator as defined in claim 4, wherein the distal tip ends of said protruder or said U-shaped elastic ring is formed with a hitching protruder, and said recess of said plate is formed with a hitching jaw.

6. The fixator as defined in claim 5, wherein said hitching jaw is formed with a hitching hole for passing through the thickness of said plate.

7. The fixator as defined in claim 1, wherein the mid-section of said plate is formed with a window-shaped hole to observe a cage between the vertebrae during surgery, and the window-shaped hole is formed with a screw thread into which a screw is threaded for fixing said cage.

8. The fixator as defined in any one of claims 1, 2 or 3, wherein said screw head is formed with a cylindrical incline so that said protruder or said U-shaped elastic ring can smoothly slide to be elastically deformed under the effect of pressure when said screws are inserted into said plate.

9. The fixator as defined in any one of claims 1, 2 or 3, wherein said protruder of said screw head fixture member or the opposite end of the distal end at said U-shaped elastic ring is fixed to said plate by rivets or by welding.

10. The fixator as defined in any one of claims 1, 2 or 3, wherein the upper surface of the screw head is grooved in the shape of a straight line or a cross, and the grooved surface is deepened towards the center thereof to form a concaved curvature so that said protruder or said U-shaped elastic ring can be deformed under the effect of pressure by the tip end of a screwdriver when the screws are installed or removed.

11. A screwdriver for rotating the screws of claim 10, wherein the distal tip end of said screwdriver for rotating said screws is protrusively formed with a protruder in the shape of a straight line or a cross for insertion into the groove, and said protruder is formed in a convex shape for being fitted into the concaved curvature.
